SEQUENCE LISTING (9 sequences)

SEQ ID NO: 1 - DNA, 1387 bp, Adonis aestivalis
gaagaacattacatggctcctgttctccttggattgaaaccaactctctccactggaagcgtcgtcaaagagactaatgtaggaagcacacttgctagtccccttaacaaaacccagaattcaagggttttggttttgggcggaacagggaaggtcggtggttccacagctttggctctctccaagttctcacctgacctcaggcttgtgattggaggtcgaaacagggagaaaggtgatgctgtagtgtctaaactaggagaaaactccgagtttgttgaagtcaacgttgacagtgtgagatctttagaatctgctctcgaagatgtggaccttgtagttcatgcagctggaccttttcaacaagcggagaagtgcactgttctagaagctgcaatatctaccaggacggcctatgtggatgtatgtgataatacaagttattccatgcaagcaaagtcttttcatgataaagcagtggctgccaacgttcctgccataacaactgctggaattttccctggagtgagcaatgtgatagcagctgagctagtgcgatcagcaagagatgaaaacactgaacctcaaagactaagattctcctattttaccgcgggttctggtggtgctggtccaacgtcgttagttactagcttcttgcttcttggtgaagaggttgttgcttacagtgaaggcgaaaaagtcgaattaaagccttatacagggaagcttaacattgacttcgggaagggagttgggaaaagagacgtttatttgtggaacttgccggaagtaagaagtggtcatgagatcttaggagtaccaactgtgagtgctcgattcggtactgcacctttcttctggaattgggcgatggtagctatgacaactctccttcctcctggtattctgagagacagaaataaaatcggaatgttggcaaattttgtgtacccttctgtacaaatttttgatgggattgcaggagaatgtcttgcaatgcgggttgatttagagtgcgcaaatgggcgcaatacttttggtatactcagtcatgaacgtctctctgtattagtgggaacttcaactgcggtgtttgctatggcaattcttgaaggaagtacgcagcctggagtttggtttccagaagagcctggagggattgcaataagtgacagagagttacttctacaacgagcatcacaaggagcgattaacttcattatgaagcagtagagtaatagattggattattcattatgtagcccagaatgacattatttacatgtaatgttgcttctatgtatcaataacataaatcacaagtcattcgtatttatataagtattcagtccatatctgggagcaaaaaaaaaaaaaaaaaaaa

SEQ ID NO: 2 - protein, 407 aa, Adonis aestivalis
MAPVLLGLKPTLSTGSVVKETNVGSTLASPLNKTQNSRVLVLGGTGKVGGSTALALSKFSPDLRLVIGGRNREKGDAVVSKLGENSEFVEVNVDSVRSLESALEDVDLVVHAAGPFQQAEKCTVLEAAISTRTAYVDVCDNTSYSMQAKSFHDKAVAANVPAITTAGIFPGVSNVIAAELVRSARDENTEPQRLRFSYFTAGSGGAGPTSLVTSFLLLGEEVVAYSEGEKVELKPYTGKLNIDFGKGVGKRDVYLWNLPEVRSGHEILGVPTVSARFGTAPFFWNWAMVAMTTLLPPGILRDRNKIGMLANFVYPSVQIFDGIAGECLAMRVDLECANGRNTFGILSHERLSVLVGTSTAVFAMAILEGSTQPGVWFPEEPGGIAISDRELLLQRASQGAINFIMKQ

SEQ ID NO: 3 - protein, 306 aa, Adonis aestivalis
AISVFSTSYSFHKNLLLHSKQDILNRPCLLFSPVVVESPMRKKKTHRAACICSVAERTRNLDIPQIEEEEENEEELIEQTDSGIIHIKKTLGGKQSRRSTGSIVAPVSCLGILSMIGPAVYFKFSRLMECGDIPVAEMGITFAAFVAAAIGTEFLSGWVHKELWHDSLWYIHKSHHRSRKGRFEFNDVFAIINALPAIALINYGFSNEGLLPGACFGTGLGTTVCGMAYIFLHNGLSHRRFPVGLIANVPYFHKLAAAHQIHHSGKFQGVPFGLFLGPQELEEVRGGTEELERVISRTAKRTQSST

SEQ ID NO: 4 - protein, 309 aa, Adonis aestivalis
MAAAISVFSSGYSFYKNLLLDSKPNILKPPCLLFSPVVIMSPMRKKKKHGDPCICSVAGRTRNLDIPQIEEEEENVEELIEQTDSDIVHIKKTLGGKQSKRPTGSIVAPVSCLGILSMIGPAVYFKFSRLMEGGDIPVAEMGITFATFVAAAVGTEFLSAWVHKELWHESLWYIHKSHHRSRKGRFEFNDVFAIINALPAIALINYGFSNEGLLPGACFGVGLGTTVCGMAYIFLHNGLSHRRFPVWLIANVPYFHKLAAAHQIHHSGKFQGVPFGLFLGPKELEEVRGGTEELERVISRTTKRTQPST

SEQ ID NO: 5 - protein, 348 aa, Adonis aestivalis
MAPVLLGLKPTLSTGSVVKETNVGSTLASPLNKTQNSRVLVLGGTGKVGGSTALALSKFSPDLRLVIGGRNREKGDAVVSKLGENSEFVEVNVDSVRSLESALEDVDLVVHAAGPFQQAEKCTVLEAAISTRTAYVDVCDNTSYSMQAKSFHDKAVAANVPAITTAGIFPGVSNVIAAGKLNIDFGKGVGKRDVYLWNLPEVRSGHEILGVPTVSARFGTAPFFWNWAMVAMTTLLPPGILRDRNKIGMLANFVYPSVQIFDGIAGECLAMRVDLECANGRNTFGILSHERLSVLVGTSTAVFAMAILEGSTQPGVWFPEEPGGIAISDRELLLQRASQGAINFIMKQ

SEQ ID NO: 6 - protein, 428 aa, Arabidopsis thaliana
MTRALLLQPYRATVRAASSRETQYDGVPEVKFSDPSRNYRVLVLGGTGRVGGSTATALSKLCPELKIVVGGRNREKGEAMVAKLGENSEFSQVDINDAKMLETSLRDVDLVVHAAGPFQQAPRCTVLEAAIKTKTAYLDVCDDTSYAFRAKSLEAEAIAANIPALTTAGIYPGVSNVMAAEMVAAARSEDKGKPEKLRFSYYTAGTGGAGPTILATSFLLLGEEVTAYKQGEKVKLRPYSGMITVDFGKGIRKRDVYLLNLPEVRSTHEVLGVPTVVARFGTAPFFWNWGMEIMTKLLPSEVLRDRTKVQQMVELFDPVVRAMDGFAGERVSMRVDLECSDGRTTVGLFSHKKLSVSVGVSTAAFVAAMLEGSTQPGVWFPEEPQGIAVEAREVLLKRASQGTFNFILNKPPWMVETEPKEVVLGIYV

SEQ ID NO: 7 - protein, 45 aa, Adonis aestivalis
MTMITPSSKLTLTKGNKSWSSTAVAAALELVDPPGCRNSHEEEHY

SEQ ID NO: 8 - DNA, 25 bp, Artificial Sequence
  Description of Artificial Sequence Synthetic primer
cacaccatggctcctgttctccttg

SEQ ID NO: 9 - DNA, 27 bp, Artificial Sequence
  Description of Artificial Sequence Synthetic primer
ctgggctacataatgaataatccaatc

(19) United States
(12) Patent Application Publication
(10) Pub. No.: US 2007/0157339 A1
(43) Pub. Date: Jul. 5, 2007

(54) Biochemical route to astaxanthin

(76) Inventor: Francis X. Cunningham JR., Chevy Chase, MD (US)

Correspondence Address: JACOBSON HOLMAN PLLC, 400 SEVENTH STREET N.W., SUITE 600, WASHINGTON, DC, 20004, US

(21) Appl. No.: 11/324,236

(22) Filed: Jan. 4, 2006

(51) Int. Cl. A01H 1/00; C07H 21/04; C12P 23/00; C12P 21/06; C12N 9/02; C12N 1/21; C12N 5/04; C12N 15/82

(52) U.S. Cl. 800/282; 435/67; 435/69.1; 435/189; 435/320.1; 435/252.33; 435/488; 435/419; 435/468; 536/23.2

(57) ABSTRACT

The sequence of a nucleic acid isolated from a cDNA library of the flowering plant Adonis aestivalis is disclosed (SEQ ID NO: 1). This DNA sequence, referred to as AdKC28, encodes for a protein that acts in conjunction with proteins encoded by either one of two other closely-related Adonis aestivalis cDNAs, AdKeto1 and AdKeto2, to convert β-carotene (β,β-carotene) into astaxanthin (3,3′-dihydroxy-4,4′-diketo-β,β-carotene). Together, these Adonis aestivalis cDNAs, when operably linked to promoters appropriate to the transgenic host, enable the production of astaxanthin and other carotenoids with 3-hydroxy-4-keto-β-rings in a variety of host cells and organisms.

This research was supported in part by the National Science Foundation, Contract No. MCB0316448. The U.S. Government has certain rights in this invention

### BACKGROUND OF THE INVENTION

The blood red color, verging on black at the base, displayed by the petals of flowers of Adonis aestivalis and Adonis annua results from the accumulation of carotenoid pigments (Egger, 1965; Neamtu et al., 1966; Seybold and Goodwin, 1959), predominantly the ketocarotenoid astaxanthin (3,3′-dihydroxy-4,4′-diketo-β,β-carotene; FIG. 1). The biosynthesis of astaxanthin occurs in a number of bacteria and fungi (Goodwin, 1980; Johnson and An, 1991), and in certain unicellular algae (Goodwin, 1980; Grung and Liaaen-Jensen, 1993; Johnson and An, 1991; Orosa et al., 2000). Astaxanthin has been found in few other plant species (Czeczuga, 1987; Goodwin, 1980), but no other species produce this ketocarotenoid in as great a quantity [ca. 1% of dry weight for the flower petals of Adonis annua according to Renstrøm et al., (1981)].

Astaxanthin has found use as a topical antioxidant (in sun blocking lotions, for example) and as an ingredient of human nutritional supplements. See U.S. Pat. No. 6,433,025 to Lorenz. This carotenoid, however, is perhaps best known for providing an attractive orange-red color to the flesh of wild salmon and other fish (Shahidi et al., 1998) and a blue hue (changing to red upon boiling as the proteins that bind astaxanthin are denatured) to the carapace of lobster and of other crustaceans (Chayen et al., 2003; Tanaka et al., 1976).

Fish and crustaceans that are raised in captivity require the addition of astaxanthin to their feed in order to acquire the appropriate coloration. The substantial and expanding market for astaxanthin as a fish feed additive is supplied largely by chemical synthesis, but there is considerable interest in the development of a biological production process to provide alternative sources of this valuable ketocarotenoid. The green alga Haematococcus pluvialis (Lorenz and Cysewski, 2000; Orosa et al., 2000) and the fungus Xanthophyllomyces dendrorhous (formerly known as Phaffia rhodozyma; Johnson, 2003; Visser et al., 2003,) have received the most attention in this regard. See also U.S. Pat. No. 6,413,736 to Jacobson et al. and incorporated by reference herein as if set forth in its entirety. The cost of producing astaxanthin biologically in these organisms remains much greater than that produced by chemical synthesis.

Currently, synthetic astaxanthin is added to feeds prepared for production of salmonids and red sea bream in aquaculture to provide a source of this carotenoid compound. See, for example, U.S. Pat. No. 5,739,006 to Abe et al. In some cases, synthetic canthaxanthin (an oxygenated carotenoid compound that is very closely related to astaxanthin) is used in place of astaxanthin in feeds for salmonids and red sea bream, but this compound does not function as well in these fishes as the naturally predominant astaxanthin.

Recently, attempts have been made, with limited success, to engineer plants for astaxanthin production by introduction of genes from algal and/or bacterial carotenoid pathways (Mann et al., 2000; Ralley et al., 2004; Stålberg et al., 2003). Some of the problems encountered with this strategy include: an incomplete conversion of precursors (β-carotene and zeaxanthin) into astaxanthin, competition of the introduced bacterial and green algal enzymes with endogenous enzymes that also use β-carotene and/or zeaxanthin as substrates (i.e. zeaxanthin epoxidase), and the accumulation of undesired intermediates of the pathway (i.e. adonixanthin and adonirubin).

Some attempts have been made to develop and exploit Adonis aestivalis as a source of astaxanthin for the pigmentation of fish (Kamata et al., 1990; Rodney, 1995), and this plant is currently grown in China expressly for this purpose. However, despite high concentrations of astaxanthin in the flower petals, a relatively low yield of petal biomass per acre makes Adonis a less than ideal vehicle for biological production of this pigment. An understanding of the biosynthetic pathway leading to astaxanthin in Adonis aestivalis would enable the pathway to be transferred to other plants, such as marigold, that could provide a much greater yield of carotenoid-containing biomass, and therefore, a much less costly source of natural astaxanthin.

From zeaxanthin (3,3′-dihydroxy-β,β-carotene), a dihydroxy carotenoid present in the green, tissues of most higher plants, the formation of astaxanthin requires only that a carbonyl be introduced at the number 4 carbon of each ring (FIG. 1). As a practical matter, the addition of the carbonyl may need to occur prior to hydroxylation of the ring [i.e. β-carotene rather than zeaxanthin would be the substrate for the enzyme, and echinenone (4-keto-β,β-carotene) and canthaxanthin (4,4′-diketo-β,β-carotene) would be the immediate products (Breitenbach et al., 1996; Fraser et al., 1998; Lotan and Hirschberg, 1995)]. Enzymes that catalyze carbonyl addition at the number 4 carbon of carotenoid β-rings have so far been identified in bacteria (De Souza et al., 2002; Harker and Hirschberg, 1999; Misawa et al., 1995a and 1995b), photosynthetic bacteria (Hannibal et al., 2000), cyanobacteria (Fernandez-Gonzalez et al., 1997; Steiger and Sandmann, 2004), and green algae (Kajiwara et al., 1995; Lotan and Hirschberg, 1995). The green algal enzymes studied are orthologs of those found in bacteria, in photosynthetic bacteria, and in certain of the cyanobacteria, as evidenced by the significant similarity of their amino acid sequences. The ketolase enzyme of the cyanobacterium Synechocystis sp. PCC6803 is distinctly different from these others (Fernandez-Gonzalez et al., 1997). It is related instead to an enzyme that catalyzes an earlier step in the carotenoid pathway of Synechocystis: the carotene isomerase (Breitenbach et al., 2001; Masamoto et al., 2001). What appears to be a third type of 4-ketolase enzyme, found in the fungus Xanthophyllomyces dendrorhous (Phaffia rhodozyma), is related to cytochrome P₄₅₀ enzymes (Hoshino et al., 2002). The activity of this enzyme has not yet been demonstrated directly. The enzyme's putative function as an “astaxanthin synthase” has been attributed on the basis of genetic complementation experiments. The gene encoding this enzyme restores the ability to synthesize astaxanthin in a X. dendrorhous mutant that accumulates only β-carotene (Hoshino et al., 2002). Because no mutants have been found that accumulate any of the intermediates between β-carotene and astaxanthin (Visser et al., 2003), it is thought that the product of this gene is responsible for both 3-hydroxylation and 4-keto addition.

The green-plant Adonis aestivalis employs an alternative way to synthesize carotenoids with 4-keto-β-rings. The present inventor has previously described (U.S. Pat. No. 6,551,807 to Cunningham) two nucleic acid sequences from Adonis aestivalis that encode enzymes (FIG. 2; SEQ ID NO: 3 and SEQ ID NO: 4) which convert β-carotene into carotenoids with ketcarotenoid-like absorption spectra (i.e. red-shifted and with a diminution of spectral fine structure). More recent work (Cunningham and Gantt, 2005) has demonstrated that the “ketolase” enzymes described in this earlier patent (AdKeto1 and AdKeto2) catalyze two different reactions: a desaturation of carotenoid β-rings at the 3-4 position and a hydroxylation at the number 4 carbon. The inventor now discloses herein the DNA sequence of an Adonis aestivalis cDNA that encodes an enzyme, referred to as AdKC28, that works in concert with either one of the two 3,4-desaturase/4-hydroxylase enzymes previously described (AdKeto1 and AdKeto2) to convert β-carotene into astaxanthin.

### SUMMARY OF THE INVENTION

There is an increasing demand for biological or “natural” sources of carotenoid pigments for use as food colorants, feed additives, and nutritional supplements. The invention described herein provides the nucleotide sequence of a cDNA (AdKC28) obtained from the flowering plant Adonis aestivalis, and entails the use of this cDNA or other nucleotides similar in sequence to this cDNA, together with either one of two Adonis aestivalis “ketolase” cDNAs (AdKeto1 and AdKeto2) disclosed in an earlier patent (U.S. Pat. No. 6,551,807 B1), to produce polypeptides that catalyze the conversion of β-carotene into astaxanthin. This invention makes available a new biochemical route, one unrelated to any previously described, that leads to the valuable ketocarotenoid astaxanthin. This new biochemical process provides a number of advantages when compared to the already existing biotechnology.

It is an object of the present invention to provide Adonis aestivalis enzymes adapted to function and efficiently produce a substantial quantity of astaxanthin in the context of a plant pathway of carotenoid biosynthesis. The production of astaxanthin in transgenic plants that express these enzymes is therefore more likely to proceed efficiently and with high yield of astaxanthin than in those wherein genes encoding bacterial or fungal or green algal enzymes are introduced.

Another object of the present invention is to provide he Adonis aestivalis genes having N-terminal sequences needed to target the membranes of the plastids efficiently in plants.

Yet another object of the present invention is to provide transgenic plants that are engineered to produce astaxanthin using genes obtained from Adonis aestivalis, itself a plant species that may be more readily accepted by consumers than transgenic plants constructed using genes isolated from bacteria or fungi or green algae. In addition, because the target tissues of transformed plants will have a striking phenotype (a dark red color), it should be possible to select for transgenic plants visually rather than with selectable markers of bacterial origin as is commonly done

It is a further object of the present invention to provide another efficient method of production of astaxanthin needing only two Adonis aestivalis gene products to convert β-carotene into astaxanthin not only in the context of a plant plastid, but also within a simple bacterial cell (see Example 1 below). Therefore, the process described in the present invention will function in cells, tissues, organs, and organisms of almost any type, as long as they accumulate or can be made to accumulate β-carotene.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings.

FIG. I illustrates the pathway to astaxanthin from b-carotene in green algae and in bacteria. Several routes may be followed, depending on the order of addition of the 3-hydroxyl and 4-keto groups to the two β-rings. Conventional numbering of the carbon atoms of a βring is shown at the lower right. Abbreviations: BKT, β-carotene 4-ketolase (Note: the bacterial β-carotene 4-ketolase enzymes are referred to as CrtW); CHYβ, β-carotene 3-hydroxylase (Note: the bacterial β-carotene 3-hydroxylase enzymes are referred to as CrtZ).

FIG. 2 shows the alignment of the amino acid sequences deduced for polypeptides encoded by Adonis aestivalis cDNAs AdKeto1 (SEQ ID NO: 3) (GenBank accession number AY644757) and AdKeto2 (SEQ ID NO: 4) (GenBank accession number AY644758). A total of 276 of 306 residues (90.2%) of the overlapping sequences (with no gaps in the alignment) are identical. These residues are shown in white text within a black box

FIG. 3 displays the nucleotide sequence of the Adonis aestivalis cDNA referred to herein as AdKC28 (SEQ ID NO: 1).

FIG. 4 displays the deduced amino acid sequence of the polypeptide encoded by AdKC28 (SEQ ID NO: 2).

FIG. 5 provides the alignment of the deduced amino acid sequence of Adonis aestivalis cDNA AdKC28 (SEQ ID NO: 5) with that deduced for an Arabidopsis thaliana gene referred to as At1g50450 (SEQ ID NO: 6) (GenBank accession number AAM19877.1 and GI:20453277). Residues identical for both sequences are shown in white text within a black box. A total of 256 of 408 residues (62.7%) of the overlapping sequences (with one gap) are identical.

FIG. 6 depicts the synthetic pathway of a 3-hydroxy-4-keto-ring catalyzed by Adonis aestivalis gene product AdKeto1 (or AdKeto2) together with AdKC28. The route used by bacteria and green algae is also shown for comparison.

### DETAILED DESCRIPTION AND PREFERRED EMBODIMENTS

The present invention is directed to a purified nucleic acid sequence that has all or some substantial portion of the nucleic acid sequence of AdKC28 (SEQ ID NO: 1), and which encodes for a protein having a particular enzymatic activity such that β-carotene is converted into astaxanthin when the polypeptide product of this nucleotide is produced together with the product of one or the other of two previously described nucleic acids (AdKeto1 and AdKeto2; SEQ ID NOS: 3 and 4; U.S. Pat. No. 6,551,807 B1).

The present invention also provides a composition comprising a purified polypeptide having all or a substantial portion of the amino acid sequence of SEQ ID NO: 2. This invention also includes the combination of the nucleic acid of SEQ ID NO: 1, or one which otherwise encodes all or a substantial portion of the polypeptide sequence of SEQ ID NO:2, together with a nucleic acid that encodes all or a substantial portion of the polypeptide of SEQ ID NO: 3 or of SEQ ID NO: 4. This invention also includes the combination of a polypeptide with all or a substantial portion of the amino acid sequence of SEQ ID NO:2, together with a polypeptide with all or a substantial portion of the amino acid sequence of SEQ ID NO: 3 or of SEQ ID NO: 4.

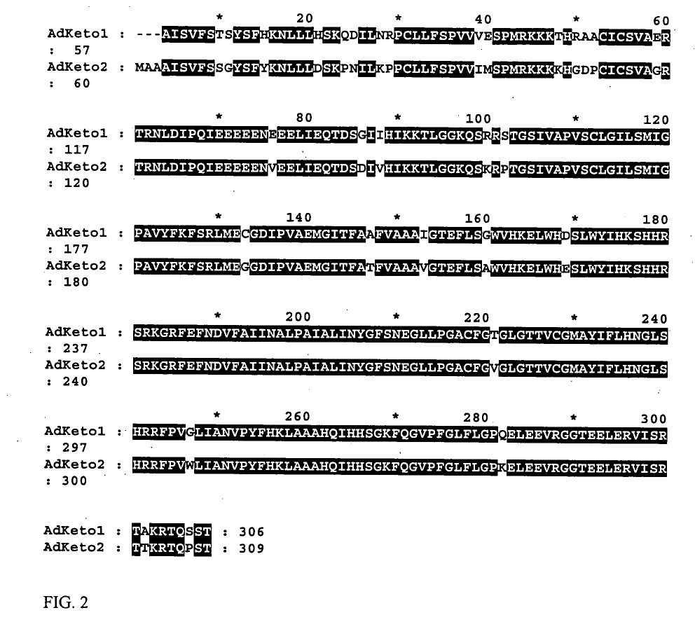

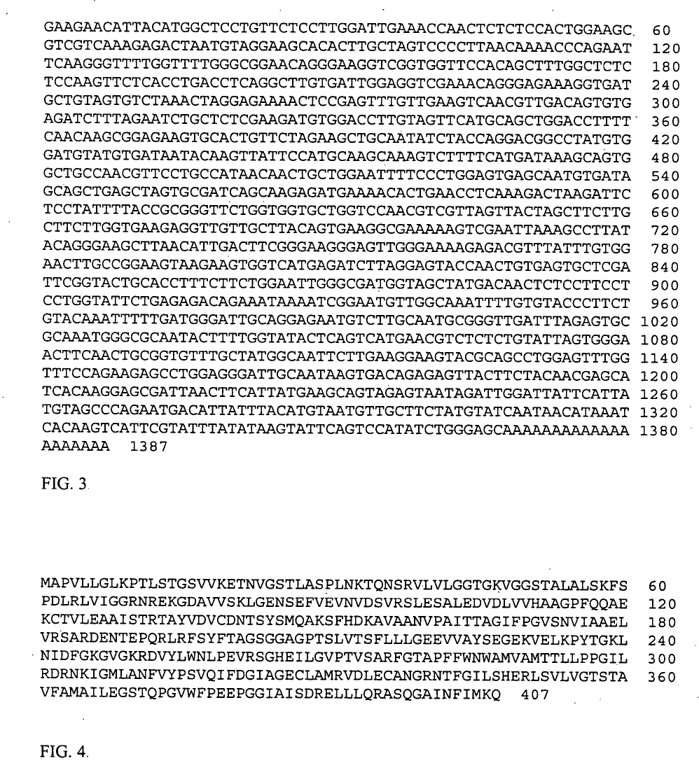

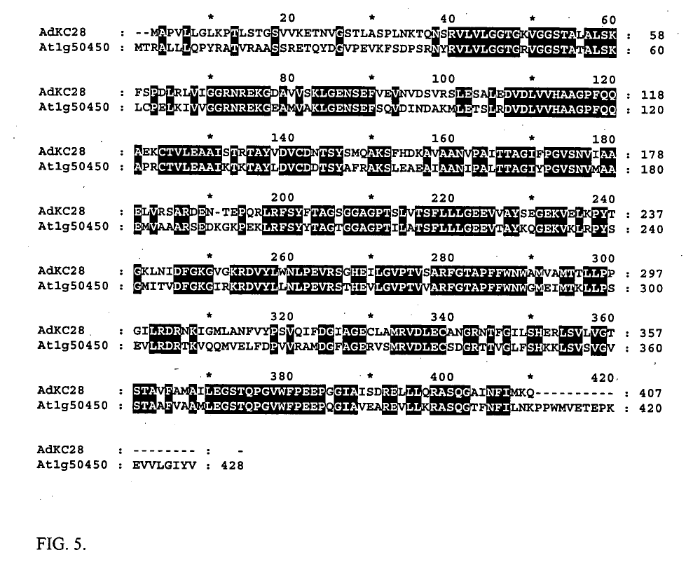

The nucleic acid sequence of Adonis aestivalis cDNA referred to as AdKC28 (SEQ ID NO: 1) is shown in FIG. 3, and the amino acid sequence deduced for the polypeptide product of this nucleic acid (SEQ ID NO: 2) is displayed in FIG. 4. No sequence in the GenBank database is more than 70% identical in amino acid sequence to AdKC28. The amino acid sequence deduced for an Arabidopsis thaliana gene/cDNA known as At1g50450 is the closest match, with only about 63% identity overall. An alignment of AdKC28 and At1g50450 is shown in FIG. 5. Genes encoding products similar in sequence to AdKC28 (SEQ ID NO: 2) are also present in many other plants (based on a BLAST search of the GenBank EST database), in the green alga Chlamydomonas reinhardtii (based on a BLAST search of the JGI Chlamydomonas reinhardtii genome database at http://genomejgi-psf.org/chlre2/chlre2.home.html) and in several cyanobacteria (ca. 30% identity for the various cyanobacterial gene products and AdKC28). The functions of the plant, algal and cyanobacterial gene products that are similar in sequence to AdKC28 are, as yet, unknown.

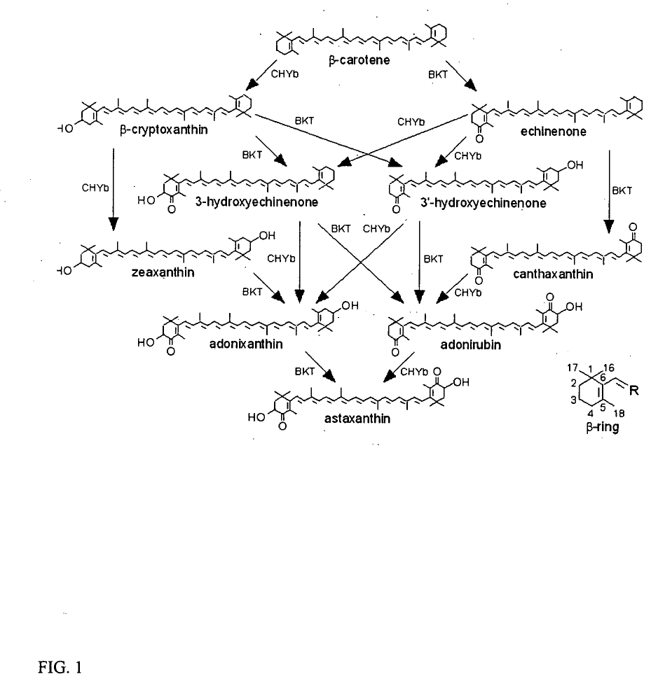

An alignment of the amino acid sequences of the products of Adonis aestivalis cDNAs AdKeto1 and AdKeto2 (SEQ ID NO: 3 and SEQ ID NO: 4) is displayed in FIG. 2. As discussed earlier, these polypeptides, which are about 90% identical in amino acid sequence overall (FIG. 2), exhibit essentially the same enzymatic activity when provided with β-carotene as the substrate, and various truncations, deletions and alterations of the coding region may be made without impairing the catalytic activity. No polypeptides presently in the GenBank database are any more than 53% identical to the amino acid sequences of the two AdKeto polypeptides (AdKeto1 and AdKeto2; SEQ ID NO: 3 and SEQ ID NO: 4).

In each case, nucleic acid and amino acid sequence similarity and identity is measured using sequence analysis software, for example, the Sequence Analysis, Gap, or BestFit software packages of the Genetics Computer Group (University of Wis. Biotechnology Center, 1710 University Avenue, Madison, Wis. 53705), MEGAlign (DNAStar, Inc., 1228 S. Park St., Madison, Wis. 53715), or MacVector (Oxford Molecular Group, 2105 S. Bascom Avenue, Suite 200, Campbell, Calif. 95008).

Conservative (i.e. similar) substitutions typically include substitutions within the following groups: glycine and alanine; valine, isoleucine and leucine; aspartic acid, glutamic acid, asparagine and glutamine; serine and threonine; lysine and arginine; and phenylalanine and tyrosine. Substitutions may also be made on the basis of conserved hydrophobicity or hydrophilicity (see Kyte and Doolittle, J. Mol. Biol. 157: 105-132 (1982)), or on the basis of the ability to assume similar polypeptide secondary structure (see Chou and Fasman, Adv. Enzymol. 47: 45-148 (1978)).

The nucleic acid molecules of the-present invention are useful for probes, primers, chemical intermediates, and in biological assays. The nucleic acid molecules are useful as a hybridization probe for messenger RNA, transcript/cDNA and genomic DNA to isolate full-length cDNA and genomic clones encoding the peptide described in FIG. 2 (SEQ ID NO: 2) and to isolate cDNA and genomic clones that correspond to variants (alleles, orthologs, etc.) producing the same or related peptides shown in FIG. 2.

A probe can correspond to any sequence along the entire length of the nucleic acid molecules provided in the Figures. Accordingly, it could be derived from 5′ noncoding regions, the coding region, and 3′ noncoding regions. However, as discussed, fragments are not to be construed as encompassing fragments disclosed prior to the. present invention.

The nucleic acid molecules are also useful as primers for PCR to amplify any given region of a nucleic acid molecule and are useful to synthesize antisense molecules of desired length and sequence.

The nucleic acid molecules are also useful for constructing recombinant vectors. Such vectors include expression vectors that express a portion of, or all of, the peptide sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations.

The nucleic acid molecules are also useful for constructing transgenic animals expressing all, or a part, of the nucleic acid molecules and peptides and are discussed in detail further.

The invention also provides vectors containing the nucleic acid molecules described herein. The term “vector” refers to a vehicle, preferably a nucleic acid molecule, which can transport the nucleic acid molecules. When the vector is a nucleic acid molecule, the nucleic acid molecules are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC. A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

Expression vectors contain cis-acting regulatory regions that are operably linked in the vector to the nucleic acid molecules such that transcription of the nucleic acid molecules is allowed in a host cell. The nucleic acid-molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, a trans-acting factor may be supplied by the host cell. Finally, a trans-acting factor can be produced from the vector itself. It is understood, however, that in some embodiments, transcription and/or translation of the nucleic acid molecules can occur in a cell-free system.

As described herein, it may be desirable to express the peptide as a fusion protein. Accordingly, the invention provides fusion vectors that allow for the production of the peptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Pharmaceutical and Nutritional Preparations

Dried Haematococcus algae, Phaffia yeast powder, or synthetic astaxanthin can be formulated into various food grade oils such as safflower, canola, tocopherols or rice bran and manufactured into gelcaps for convenient ingestion. Alternatively, dried Haematococcus algae, Phaffia yeast powder, or synthetic astaxanthin can be stabilized by various commercial processes and added directly to foods or beverages.

The carotenoid astaxanthin has never been suggested as a dietary supplement to retard or prevent sunburns or related cancers. Nor have the combined properties of astaxanthin as a potent antioxidant and an immune system modulator been previously recognized or proposed as a dietary supplement to retard or prevent sunburns.

Thus, the inventor also presents a treatment and method for retarding and prevention of sunburns, and possibly related cancers resulting from long term sunburn damage and a treatment and method of retarding and preventing sunburns by administering a therapeutically effective dose of astaxanthin made using the enzyme derived from the DNA sequence AdKC28.

The astaxanthin made using the enzyme derived from the DNA sequence AdKC28 is preferably administered orally, in doses of between about 1 to about 100 mg per day. Doses of between about 2 to about 10 mg per day are preferable.-The dose may be administered to be taken with meals, twice daily.

In addition to an oral administration, a formulation of astaxanthin may also be applied in a cream or injected into the exposed area. Such a dose would also be in the range of about 1 to 100 mg per day.

It is preferable, with an ingestible form of astaxanthin, to begin administering the astaxanthin at least two or three days before sun exposure, and preferably at least a week before exposure, in order to prevent sunburn. However, as seen below in the examples, even ingestion during or after exposure provides beneficial effects. With the topical and injectable treatment, astaxanthin may be administered before, during, or after exposure.

Any and all organisms that synthesize carotenoids are potential candidates for astaxanthin production using the Adonis aestivalis cDNAs disclosed and described herein. A number of plants, some fungi and yeasts, and several green algae have been utilized commercially as sources of carotenoid pigments. In these organisms the carotenoids of interest may be accumulated within specific organs or tissues (e.g. the flower petals of marigold, the roots of carrot and the tubers of sweet potato), may be induced under particular environmental conditions or times of development (as in certain species of the green algae Haematococcus and Dunaliella), or may result from transgenic modification of the host (as in the seeds of canola expressing a bacterial phytoene synthase gene; Ravanello et al., 2003; Shewmaker et al., 1999).

Host systems according to the present invention preferably comprise any organism which is capable of producing carotenoids, or which already produces carotenoids. Such organisms include plants, algae, certain bacteria, cyanobacteria and other photosynthetic bacteria. Transformation of these hosts with vectors according to the present invention can be done using standard techniques. See, for example, Sambrook et al., Molecular Cloning A Laboratory Manual, Cold Spring Harbor Laboratory, Cold Spring Harbor, N.Y., 1989; Ausubel et al., Current Protocols in Molecular Biology, Greene Publishing and Wiley Interscience, New York, 1991.

The present invention also includes vectors containing the nucleic acids of the invention. Suitable vectors according to the present invention comprise a gene encoding a ketolase enzyme as described above, wherein the gene is operably linked to a suitable promoter. Suitable promoters for the vector can be constructed using techniques well known in the art (see, for example, Sambrook et al., Molecular Cloning A Laboratory Manual, Cold Spring Harbor Laboratory, Cold Spring Harbor, N.Y., 1989; Ausubel et al., Current Protocols in Molecular Biology, Greene Publishing and Wiley Interscience, New York, 1991). Suitable vectors for eukaryotic expression in plants are described in Fray et al., (1995; Plant J. 8:693-701) and Misawa et,al, (1994; Plant J. 6:481-489). Suitable vectors for prokaryotic expression include pACYC184, pUC 119, and pBR322 (available from New England BioLabs, Bevery, Mass.) and pTrcHis (Invitrogen) and pET28 (Novagen) and derivatives thereof. The vectors of the present invention can additionally contain regulatory elements such as promoters, repressors, selectable markers such as antibiotic resistance genes, etc., the construction of which is very well known in the art.

For the purpose of astaxanthin production of the present invention, the preferred microbial, fungal, plant and algal hosts for the Adonis aestivalis genes are those that produce or can be made to produce a substantial quantity of β-carotene or metabolites thereof. Among the more preferred hosts at this time are: marigold (in the flowers; especially those of mutants or varieties that accumulate predominantly b-carotene), transgenic canola (with carotenoid-accumulating seeds, as in Shewmaker et al., 1999), oil palm (various species of the genus Elaeis; the carotenoid-accumulating seeds), carrot (the β-carotene-accumulating root), sweet potato (the β-carotene-rich tubers), maize (the carotenoid-accumulating seeds), tomato (the fruits, especially in varieties or transgenic plants that accumulate largely β-carotene rather than lycopene), and various high β-carotene producing species of the green alga Dunaliella.

The genes encoding the ketolase enzymes as described above, when cloned into a suitable expression vector, can be used to overexpress these enzymes in a host cell expression system or to inhibit the expression of these enzymes. For example, a vector containing a gene of the invention may be used to increase the amount of ketocarotenoids *in an organism and thereby alter the nutritional or commercial value or pharmacology of the organism. A vector containing a gene of the invention may also be used to modify the carotenoid production in an organism.

Methodologies for producing transgenic bacteria, fungi, algae, and plants are widely known and familiar to those skilled in the arts. It is desirable to employ promoters that restrict the expression of the Adonis genes to the carotenoid-rich tissues or to an appropriate time of development in order to avoid possible adverse effects on yield.

Therefore, the present invention includes a method of producing a ketocarotenoid in a host cell, the method comprising inserting into the host cell a vector comprising a heterologous nucleic acid sequence which encodes for a protein having ketolase enzyme activity and comprises (1) SEQ ID NO: 1 or 3 or (2) a sequence which encodes the amino acid sequence of SEQ ID NO: 2 or 4, wherein the heterologous nucleic acid sequence is operably linked to a promoter; and expressing the heterologous nucleic acid sequence, thereby producing the ketocarotenoid.

Oh the basis of the teachings disclosed here and in an earlier patent (U.S. Pat. No. 6,551,807, hereby incorporated by reference in its entirety as if completely set forth in the specification), one of ordinary skill in the art would be able create nucleotides that encode polypeptides similar in sequence to and with the same catalytic activity as AdKC28, AdKeto1 and AdKeto2. One can isolate such nucleotides from a different accession of Adonis aestivalis or from one of the other species of Adonis that produce astaxanthin. Alternatively, one skilled in the art can create different nucleotides that would encode the polypeptides of SEQ ID NO: 2, SEQ ID NO: 3, and SEQ ID NO: 4, or polypeptides a bit different from SEQ ID NO: 2, SEQ ID NO: 3, and SEQ ID NO: 4 that would retain the catalytic activity of these proteins. Such modifications are well known in genetic engineering, such as whether to introduce a restriction site, add a transit sequence, make “conservative” (i.e. similar) substitutions of various amino acids, or alter the codon usage to be more compatible with the host organism. Therefore, in the context of the present invention, the Applicants disclose and claim nucleotides that encode polypeptides that are >70% identical to, in whole or in large part, and exhibit the catalytic function of those polypeptides of SEQ ID NO: 2, SEQ ID NO: 3, and SEQ ID NO: 4. Such claims would not include or encompass any nucleotides or polypeptides that are currently available in the GenBank databases.

The term “modifying the production” means that the amount of carotenoids produced can be enhanced, reduced, or left the same, as compared to an untransformed host cell. In accordance with one embodiment of the present invention, the make-up of the carotenoids (i.e., the type of carotenoids produced) is changed vis a vis each other, and this change in make-up may result in either a net gain, net loss, or no net change in the amount of carotenoids produced in the cell.

It is expressly stated that the numbering of the elements of the sequences (on one hand nucleic acid sequence and on the other amino acid sequence) shall not be understood as a fixed or limiting definition. The numbering shall merely provide the information of the positions of the sequence elements to each other in relative terms and is therefore a reference.

The term “derivative” means, within the context of the present invention, that the sequences of these molecules differ from the sequences of the nucleic acid molecules according to the invention or to be suitably employed in accordance with the invention in one or more positions and exhibit a high degree of homology to these sequences. Homology means a sequential identity of at least 60%, preferably over 70%, and especially preferably over 85%, in particular over 90% and very especially preferably over 95%. The deviations relative to the nucleic acid molecules according to the invention or to the nucleic acid molecules to be suitably employed in accordance with the invention may have originated by means of one or more deletions, substitutions, insertions (addition) or recombinations.

Furthermore, homology means that a functional and/or structural equivalence exits between the nucleic acid molecules in question and the proteins encoded by them. The nucleic acid molecules which are homologous to the molecules according to the invention or to the molecules to be suitably employed in accordance with the invention and which constitute derivatives of these molecules are, as a rule, variations of these molecules which constitute modifications which exert the same, a virtually identical or a similar biological function. They maybe naturally occurring variations, for example sequences from other plant species, or mutations, it being possible for these mutations to have occurred naturally or to have been introduced by directed mutagenesis. The variations may further be synthetic sequences. The allelic variants may be naturally occurring variants or else synthetic variants or variants generated by recombinant DNA technology.

The term “part” regarding the nucleic acid molecule encoding an AdKC28 protein according to instant invention encompasses a poly- or oligonucleotide consisting of about at least 30-99, preferably at least 100, more preferably at least 200, in particular at least 300, and most preferably at least 400 of the nucleotides of the nucleic acid molecule encoding an AdKC28 protein or derivative thereof according to the invention. The term “part” is not limited to portions of the nucleic acid molecules which are long enough to encode a functionally active portion of the AdKC28 protein as described.

Having generally described this invention, a further understanding can be obtained by reference to the following specific example which is provided herein for the purpose of illustration only. It is not intended that this example be limiting.

### EXAMPLE 1

Production of Astaxanthin in the Bacterium Escherichia coli: a Case Study

A strain of the common laboratory bacterium E. coli was engineered to produce the carotenoid β-carotene by introduction of a plasmid (pAC-BETA) containing the requisite genes from the bacterium Erwinia herbicola (Cunningham et al., 1996). Introduction of a second plasmid containing either the Adonis aestivalis DNA sequence AdKeto1 or AdKeto2 resulted in the conversion of b-carotene into several other carotenoids that contain β-rings with a desaturation at the 3-4 position and/or an hydroxyl group at the number 4 carbon (Cunningham and Gantt, 2005). Addition of a third plasmid, containing the Adonis aestivalis DNA sequence AdKC28, resulted in the synthesis and accumulation, predominantly, of the ketocarotenoid astaxanthin. Absent the second plasmid that contained either AdKeto1 or AdKeto2, the introduction of the plasmid containing the Adonis aestivalis DNA sequence AdKC28 into the β-carotene accumulating E. coli strain did not alter the carotenoid content: b-carotene remained the predominant pigment.

Two different versions of the third plasmid were used in the above experiments, with each resulting in the accumulation of astaxanthin in good yield. In one plasmid the AdKC28 cDNA was fused in frame to a portion of a gene encoding the N terminus of the lacZ polypeptide (in plasmid vector pBluescript SK-; from Stratagene Cloning Systems). The amino acid sequence of the fusion protein specified by this chimerical gene consisted of the full length ADKC28 (SEQ ID NO: 2) with additional N terminal sequence specified by lacZ and the 5′ untranslated region of AdKC28

(SEQ ID NO: 7)

(MTMITPSSKLTLTKGNKSWSSTAVAAALELVDPPGCRNSHEEEHY).

A second version of the plasmid containing AdKC28 was constructed so as to produce the authentic full length polypeptide (SEQ ID NO: 2) under control of the tightly-regulated bacterial araBAD promoter. The coding region of AdKC28 was amplified by PCR using oligonucleotide primers AdKC28Nco-N (CACACCATGGCTCCTGTTCTCCTTG) (SEQ ID NO: 8) and AdKC28-C (CTGGGCTACATAATGAATAATCCAATC) (SEQ ID NO: 9), and the PCR product was digested with the appropriate restriction enzymes and ligated in the NcoI and XhoI sites of plasmid pBAD/HisB (Invitrogen). Biosynthesis of astaxanthin with this third plasmid (in E. coli cultures also containing plasmids pAC-BETA and pAdKeto1 or pAdKeto2) occurred only when arabinose was added to induce expression of AdKC28 from the araBAD promoter.

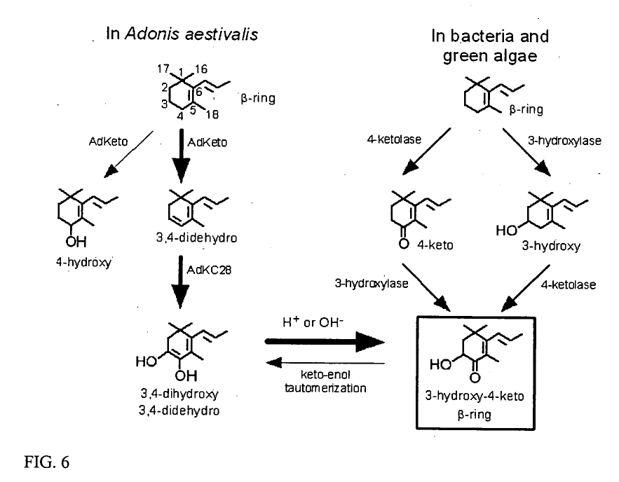

From the above results it can be deduced that, unexpectedly and in contrast to the pathways of bacteria and green algae, the route to a 3-hydroxy-4-keto-β-ring in carotenoids of Adonis aestivalis does not proceed via either a 3-hydroxy-β ring or a 4-keto-β ring. The sequence of reactions of the present invention (FIG. 6) includes first a desaturation of the β-ring at the 3,4 position (a reaction catalyzed by the AdKeto 1 and AdKeto2 “ketolase” enzymes; Cunningham and Gantt, 2005). This is then followed by a dihydroxylation at the number 3 and 4 carbons (a reaction catalyzed by the product of Adonis aestivalis cDNA AdKC28), with the 3,4-desaturation either retained or reintroduced by AdKeto1 or AdKeto2. The 3,4-didehydro-3,4-dihydroxy-β-ring thereby produced will then spontaneously convert to a 3-hydroxy-4-keto-β-ring as a consequence of keto-enol tautomerization.

The data clearly demonstrate that the products of two cDNAs derived from mRNA isolated from a flowering plant, Adonis aestivalis, are sufficient to convert β-carotene into the valuable ketocarotenoid astaxanthin in the context of a simple bacterial cell. The same two gene products, therefore, should prove sufficient to convert β-carotene into astaxanthin in a wide variety of host organisms, both prokaryotic and eukaryotic, and both photosynthetic and nonphotosynthetic.

Having described the invention, many modifications thereto will become apparent to those skilled in the art to which it pertains without deviation from the spirit of the invention as defined by the scope of the appended claims.

### REFERENCES

The references cited in the above specification, along with the following references, are incorporated by reference in their entireties as if fully set forth in the specification:
Breitenbach, J., Misawa, N., Kajiwara, S. and Sandmann, G. (1996) Expression in Escherichia coli and properties of the carotene ketolase from Haematococcus pluvialis. FEMS Microbiol. Lett. 140, 241-246.
Breitenbach, J., Vioque, A. and Sandmann, G. (2001) Gene sll0033 from Synechocystis 6803 encodes a carotene isomerase involved in the biosynthesis of all-E lycopene. Z. Naturforsch. [C]. 56, 915-917.
Chayen, N. E., Cianci, M., Grossmann, J. G., Habash, J., Helliwell, J. R., Nneji, G. A., Raftery, J., Rizkallah, P. J. and Zagalsky, P. F. (2003) Unravelling the structural chemistry of the colouration mechanism in lobster shell. Acta Crystallographica D. Biological Crystallography 59, 2072-2082.
Choi S.-K., Nishida, Y., Matsuda, S., Adachi, K., Kasai, H., Peng, X., Komemushi, S., Miki, W. and Misawa, N. (2005) Characterization of β-carotene ketolases, CrtW, from marine bacteria by complementation analysis in Escherichia coli. Mar. Biotechnol. July 5; [Epub ahead of print].
Cunningham, F. X., Jr. and E. Gantt (2005) A study in scarlet: enzymes of ketocarotenoid biosynthesis in the flowers of Adonis aestivalis. Plant J. 41, 478-92.
Cunningham, F. X. Jr., Pogson, B., Sun, Z., McDonald, K. A., DellaPenna, D. and Gantt, E. (1996) Functional analysis of the beta and epsilon lycopene cyclase enzymes of Arabidopsis reveals a mechanism for control of cyclic carotenoid formation. Plant Cell 8, 1613-1626.
Czeczuga, B. (1987) Ketocarotenoids—autumn carotenoids in Metasequoia glyptostroboides. Biochem. Syst. Ecol. 15, 303-306.
De Souza, M. L., Kollmann, S. R. and Schroeder, W. A. (2002) Carotenoid Biosynthesis. International patent application PCT WO/02/079395-B.
Egger, K (1965) Die Ketocarotinoide in Adonis annua L. Phytochemistry 4, 609-618.
Fernandez-Gonzalez, B. F., Sandmann, G. and Vioque, A. (1997) A new type of asymmetrically acting beta-carotene ketolase is required for the synthesis of echinenone in the cyanobacterium Synechocystis sp. PCC 6803. J. Biol. Chem. 272, 9728-9733.
Fraser, P. D., Shimada, H., and Misawa, N. (1998) Enzymic confirmation of reactions involved in routes to astaxanthin formation, elucidated using a direct substrate in vitro assay. Eur. J. Biochem. 252, 229-236.
Goodwin, T. W. (1980) The Biochemistry of the Carotenoids 2^{nd} edn, Vol. 1. London: Chapman and Hall.
Grung, M. and Liaaen-Jensen, S. (1993) Algal carotenoids 52; secondary carotenoids of algae 3; carotenoids in a natural bloom of Euglena sanguinea. Biochem. Syst. Ecol. 21, 757-763.
Hannibal, L., Lorquin, J., D'Ortoli, N. A., Garcia, N., Chaintreuil, C., Masson-Boivin, C., Dreyfus, B. and Giraud, E. (2000) Isolation and characterization of canthaxanthin biosynthesis genes from the photosynthetic bacterium Bradyrhizobium sp. Strain ORS278. J. Bacteriol. 182, 3850-3853.
Harker, M. and Hirschberg, J. (1999) Carotenoid biosynthesis genes,in the bacterium Paracoccus marcusii MH1, unpublished. GenBank Accession Number Y15112.
Hoshino, T., Kazuyuki, O. and Setoguchi, Y. (2002) Astaxanthin synthase. U.S. Pat. No. 6,365,386 B1.
Johnson, E. A. (2003) Phaffia rhodozyma: colorful odyssey. Int. Microbiol. 6, 169-174.
Johnson, E. A. and An, G. H. (1991) Astaxanthin from microbial sources. Crit. Rev. Biotechnol. 11, 297-326.
Kajiwara, S., Kakizono, T., Saito, T., Kondo, K., Ohtani, T., Nishio, N., Nagai, S. and Misawa, N. (1995) Isolation and functional identification of a novel cDNA for astaxanthin biosynthesis from Haematococcus pluvialis, and astaxanthin synthesis in Escherichia coli. Plant Mol. Biol. 29, 343-352.
Kamata, T., Tanaka, Y., Yamada, S. and Simpson K. L. (1990) Study of carotenoid composition and fatty-acids of astaxanthin diester in rainbow-trout salmo-gairdneri fed the Adonis extract. Nippon Suisan Gakkaishi 56, 789-794.
Lorenz, R. T. and Cysewski, G. R. (2000) Commercial potential for Haematococcus microalgae as a natural source of astaxanthin. Trends Biotechnol. 18, 160-167.
Lotan, T. and Hirschberg, J. (1995) Cloning and expression in Escherichia coli of the gene encoding beta-C-4-oxygenase, that converts beta-carotene to the ketocarotenoid canthaxanthin in Haematococcus pluvialis. FEBS Lett. 364, 125-128.
Mann, Y., Harker, M., Pecker, I. and Hirschberg, J. (2000) Metabolic engineering of astaxanthin production in tobacco flowers. Nat. Biotechnol. 18, 888-892.
Masamoto, K., Wada, H., Kaneko, T. and Takaichi, S. (2001) Identification of a gene required for cis-to-trans carotene isomerization in carotenogenesis of the cyanobacterium Synechocystis sp. PCC 6803. Plant Cell Physiol. 42, 1398-1402.
Misawa, N., Satomi, Y., Kondo, K., Yokoyama, A., Kajiwara, S., Saito, T., Ohtani, T. and Miki, W. (1995a) Structure and functional analysis of a marine bacterial carotenoid biosynthesis gene cluster and astaxanthin biosynthetic pathway proposed at the gene level. J. Bacteriol. 177, 6575-658418.
Misawa, N., Kajiwara, S., Kondo, K, Yokoyama, A., Satomi, Y., Saito, T., Miki, W. and Ohtani, T. (1995b) Canthaxanthin biosynthesis by the conversion of methylene to keto groups in a hydrocarbon beta-carotene by a single gene. Biochem. Biophys. Res. Commun. 209, 867-876.
Neamtu, G., Tamas, V. and Bodea, C. (1966) Die carotinoide aus Einigen Adonis-arten. Rev. Roum. Biochem. 3, 305-310.
Orosa, M., Torres, E., Fidalgo, P. and Abalde, J. (2000) Production and analysis of secondary carotenoids in green algae. J. Appl. Phycol. 12, 553-556.
Ralley, L., Enfissi, E. M. A., Misawa, N., Schuch, W., Bramley, P. M. and Fraser, P. D. (2004) Metabolic engineering of ketocarotenoid formation in higher plants. Plant J. 39, 477-486.
Ravanello, M. P., Ke, D., Alvarez, J., Huang, B. and Shewmaker, C. K. (2003) Coordinate expression of multiple bacterial carotenoid genes in canola leading to altered carotenoid production. Metabolic Eng. 5, 255-263.
Renstrøm, B., Berger, H. and Liaaen-Jensen, S. (1981) Esterified, optically pure (3S, 3′S)-astaxanthin from flowers of Adonis annua. Biochem. Syst. Ecol. 9, 249-250.
Rodney, M. (1995) Astaxanthin from flowers of the genus Adonis. U.S. Pat. No. 5,453,565.
Seybold, A. and Goodwin, T. W. (1959) Occurrence of astaxanthin in the flower petals of Adonis annua L. Nature 184, 1714-1715.
Shahidi, F., Metusalach and Brown, J. A. (1998) Carotenoid pigments in seafoods and aquaculture. Crit. Rev. Food Sci. Nutrition 38, 1-67.
Shewmaker, C. K, Sheehy, J. A., Daley, M., Colburn, S. and Ke, D. Y. (1999) Seed-specific overexpression of phytoene synthase: increase in carotenoids and other metabolic effects. Plant J. 20, 401-412.
Stålberg, K, Lindgren, O., Ek, B. and Höglund, A.-S. (2003) Synthesis of ketocarotenoids in the seed of Arabidopsis thaliana. Plant J. 36, 771-779.
Steiger, S. and Sandmann, G. (2004) Cloning of two carotenoid ketolase genes from Nostoc punctiforme for the heterologous production of canthaxanthin and astaxanthin. Biotechnol. Lett. 26, 813-817.
Tanaka, Y., Matsuguchi, H., Katayama, T., Simpson, K. L. and Chichester, C. O. (1976) The biosynthesis of astaxanthin-XVI. The carotenoids in Crustacea. Comp. Biochem. Physiol. B. 54, 391-393.
Visser, H., van Ooyen, A. J. J. and Verdoes, J. C. (2003) Metabolic engineering of the astaxanthin-biosynthetic pathway of Xanthophyllomyces dendrorhous. FEMS Yeast Res. 4, 221-231.

I claim:

1. A purified nucleic acid sequence of having the nucleotide sequence of SEQ ID NO: 1.

2. An isolated polypeptide encoded by nucleotide sequence of claim 1.

3. The polypeptide of claim 2 having the amino acid sequence of SEQ ID NO:2.

4. The purified nucleic acid sequence of claim 1 or a substantial portion thereof, which encodes for a polypeptide that works in conjunction with AdKeto1 or AdKeto2 to convert β-carotene into astaxanthin.

5. A purified nucleic acid sequence which encodes for a polypeptide that is 90% or more identical in amino acid sequence to that of SEQ ID NO: 2, or a substantial portion thereof, that works in conjunction with AdKeto1 or AdKeto2 to convert β-carotene into astaxanthin.

6. A purified nucleic acid sequence which encodes for a polypeptide that is 70% or more identical in amino acid sequence to that of SEQ ID NO: 2 or a substantial portion thereof, that works in conjunction with AdKeto1 or AdKeto2 to convert β-carotene into astaxanthin.

7. A vector that contains the nucleic acid sequence of claim 4.

8. A vector that contains the nucleic acid sequence of claim 5.

9. A vector that contains the nucleic acid sequence of claim 6.

10. A purified polypeptide comprising the amino acid sequence of SEQ ID NO: 2 or a substantial portion thereof, that works in conjunction with AdKeto1 or AdKeto2 to convert β-carotene into astaxanthin.

11. A purified-polypeptide comprising an amino acid sequences that is 90% or more identical in amino acid sequence to that of SEQ ID NO: 2 or a substantial portion thereof, that works in conjunction with AdKeto1 or AdKeto2 to convert β-carotene into astaxanthin.

12. A purified polypeptide comprising an amino acid sequence that is 70% or more identical in amino acid sequence to that of SEQ ID NO: 2 or a substantial portion thereof, that works in conjunction with AdKeto1 or AdKeto2 to convert β-carotene into astaxanthin.

13. A recombinant, double-stranded DNA molecule comprising:
a) a promoter functional in plant cells, and
b) the DNA sequence of SEQ ID NO:1 encoding for a protein having enzyme activity which converts the enzymatic product of either AdKeto1 or AdKeto2 to astaxanthin, wherein said DNA sequence is operatively linked to the promoter in sense orientation.

14. The DNA molecule according to claim 13, wherein the prokaryotic organism is Escherichia coli.

15. The DNA molecule according to claim 13, wherein the DNA sequence is a sequence which hybridizes with the coding region depicted as SEQ ID NO. 1 under conditions wherein sodium chloride concentrations are about 0.02 M to about 0.15 M and temperatures range from about 50° C. to about 70° C.

16. The DNA molecule according to claim 13, wherein the DNA sequence has at least about 80% identity with the coding region depicted as SEQ ID NO. 1.

17. The DNA molecule according to claim 13, wherein the DNA sequence has the coding region depicted as SEQ ID NO. 1, or a nucleotide sequence that encodes the same peptide as SEQ ID NO. 1.

18. A transgenic plant cell containing in its genome a recombinant DNA molecule according to claim 13.

19. A transgenic plant containing plant cells according to claim 18.

20. A vector which comprises the nucleic acid sequence of SEQ ID NO: 1, wherein the nucleic acid sequence is operably linked to a promoter.

21. A host cell which is transformed with the vector of claim 20.

22. The host cell of claim 21, wherein the host cell is selected from the group consisting of a bacterial cell, an algal cell and a plant cell.

23. The host cell of claim 21, wherein the host cell is a photosynthetic cell.

24. The host cell of claim 21, wherein the host cell contains a ketocarotenoid.

25. The host cell of claim 21, wherein the host cell contains modified levels of carotenoids, relative to an untransformed host cell.

26. A method of producing astaxanthin and other carotenoids with 3-hydroxy-4-keto-β-rings in a host cell, the method comprising inserting into the host cell nucleic acid sequences that encode all or substantial portions of AdKC28 (SEQ ID NO: 2) and AdKeto1 (SEQ ID NO: 3) or AdKeto2 (SEQ ID NO: 4).

27. The method of claim 26, wherein the nucleotide sequences encode polypeptides that are 90% or more identical in sequence to all or substantial portions of SEQ ID NO: 2 and SEQ ID NO: 3 or SEQ ID NO: 4.

28. The method of claim 26, wherein the nucleotide sequences encode polypeptides that are 70% or more identical in sequence to all or substantial portions of SEQ ID NO: 2 and SEQ ID NO: 3 or SEQ ID NO: 4.

29. The method of claim 26, wherein the host cell is a bacterium, an archaea, an alga, a yeast, a fungus or a plant.

30. The method of claim 27, wherein the host cell is a bacterium, an archaea, an alga, a yeast, a fungus or a plant.

31. The method of claim 28, wherein the host cell is a bacterium, an archaea, an alga, a yeast, a fungus or a plant.

32. A nutrient additive for aquatic organisms comprising astaxanthin made using the method of claim 29.

33. The nutrient additive of claim 32, wherein the aquatic organisms are selected from the group consisting of: salmon, shrimp, crabs and lobster.

34. A sunscreen composition for retarding or prevent sunburns of the skin, comprising about 1 to 100 mg of astaxanthin per day, in a formulation comprising astaxanthin as the single active ingredient, administered to a patient in need thereof, orally, topically, or by injection, wherein the astaxanthin is made using the method of claim 29.

35. The composition according to claim 34, comprising about 2-10 mg of astaxanthin per day administered to said patient, orally, topically, or by injection.

36. A method for retarding or prevent sunburns of the skin in a human, comprising the steps of administering about 1 to 100 mg of astaxanthin per day, in a formulation comprising astaxanthin as the single active ingredient, administered to a patient in need thereof, orally, topically, or by injection, wherein the astaxanthin is made using the method of claim 29; and assessing whether there has been any sunburn on the skin.
